(19) United States
(12) Patent Application Publication
(10) Pub. No.: US 2020/0338305 A1
(43) Pub. Date: Oct. 29, 2020

(54) CATHETER

(71) Applicant: KANEKA CORPORATION, Osaka (JP)

(72) Inventor: Hajime SAKATA, Tokyo (JP)

(73) Assignee: KANEKA CORPORATION, Osaka (JP)

(21) Appl. No.: 16/839,119

(22) Filed: Apr. 3, 2020

(30) Foreign Application Priority Data
Apr. 26, 2019 (JP) 2019-086532

(51) Int. Cl. A61M 25/00; A61M 25/01; A61N 1/05; A61B 5/042; A61B 5/00

(52) U.S. Cl. CPC A61M 25/005; A61M 25/0136; A61M 25/0147; A61B 5/042; A61B 5/6852; A61N 1/0587

(57) ABSTRACT

The catheter handle has a handle body 20 connected to the proximal side of the catheter tube, a rotary member 30 fixed to a proximal portion of the wire 50 and disposed in the handle body 20, a tubular member 12 having the wire 50 inside and extending from the catheter tube to the handle body 20, a contact member 60 having a contact part with the wire 50, a distance between the most distal contact point 70 and the central axis is less than half of an inner diameter of the tubular member 12.

### CROSS-REFERENCE TO RELATED APPLICATIONS

The present application claims priority based on Japanese Patent Application No. 2019-86532 filed on Apr. 26, 2019. All the contents described in Japanese Patent Application No. 2019-86532 filed on Apr. 26, 2019 are incorporated herein by reference.

### TECHNICAL FIELD

The present invention relates to a catheter including a catheter handle.

### BACKGROUND ART

A medical catheter is generally composed of a catheter tube inserted to body lumens such as a blood vessel, a digestive tube, and a urinary duct, and a handle disposed on the proximal side of the catheter tube. A catheter has been known that is configured such that the distal side of the catheter tube can be bent by manipulating the handle. In the catheter tube, such a catheter has one or two wires the distal portion of which is fixed to the distal side of the catheter tube and the proximal portion of which is connected to the handle. Pulling the wires toward the handle by manipulating the handle enables the distal side of the catheter tube to bend. As a structure of making the catheter tube bend, a method is known where the handle has a wire locking part and a rotation part, and the position of the wire locking part is changed by rotating the rotating part to pull the wire toward the handle. The wires, which are thin and receives pulling force repeatedly, are required to be tough not so as to be damaged or broken with repeated manipulations.

Patent Document 1 discloses an electrode catheter in which wires and conductive wires extends through a catheter tube and the wire is led to the outside of the catheter tube via a side opening located on the side of the catheter tube. The catheter tube of the Patent Document 1 has high strength to secure a pass for the wires, which enables the wire not to interfere the conductive wires. Patent Document 2 discloses a catheter in which wires extend in the direction of a handle through a sheath, which enables the wires not to cut in a wall of the middle part of the catheter when the middle part of the catheter is made bent.

### RELATED ART DOCUMENTS

### Patent Documents

Patent Document 1: JP-A-2018-143603

Patent Document 2: JP-A-2013-106952

### SUMMARY OF THE INVENTION

### Problems to be Solved by the Invention

While the catheters disclosed in the Patent Document 1 and the Patent Document 2 can prevent the wires from interfering the conductive wires in the catheter tube, or prevent the wires from cutting in the wall of the catheter tube, they do not focused on damage or breaking of the wires in the handle at all. In realizing the above situation, the purpose of the present invention is to offer a catheter that can make a wire less likely to be damaged or broken and control the degree of bending of a catheter tube at the distal side by manipulating a catheter handle connected to the catheter tube.

### Means for Solving the Problems

The present invention that can solve the above problem is as follows:

A catheter, comprising,

a catheter tube having a distal side and a proximal side and provided with at least one wire therein, and

a catheter handle connected to the proximal side of the catheter tube, the catheter handle comprising,

a handle body connected to the proximal side of the catheter tube,

a rotary member fixed to a proximal portion of the wire and disposed in the handle body,

a tubular member having the wire inside and extending from the catheter tube to the handle body,

a contact member having a contact part with the wire and located more proximal than a proximal end of the tubular member, wherein

in a plane that is orthogonal to a central axis of the tubular member and includes a most distal contact point among the contact part, a distance [d] between the most distal contact point and the central axis satisfies the following equation (1) in terms of an inner diameter of the tubular member [dt].

[d]<[dt]/2 (1)

The contact member is preferably located more distal than a distal end of the rotary member.

The wire preferably comprises a first wire and a second wire,

the contact member has a first contact part with the first wire and a second contact part with the second wire,

in a plane that is orthogonal to the central axis of the tubular member and includes a most distal first contact point among the first contact part, a distance [d] between the most distal first contact point and the central axis preferably satisfies the above equation (1) in terms of the inner diameter of the tubular member [dt], and

in a plane that is orthogonal to the central axis of the tubular member and includes a most distal second contact point among the second contact part, a distance [d] between the most distal second contact point and the central axis preferably satisfies the above equation (1) in terms of the inner diameter of the tubular member [dt].

A distance [dw] between the first wire and the second wire at a proximal end of the tubular member is preferably smaller than the inner diameter of the tubular member [dt].

The contact member is preferably a hollow body having a single hollow, and the minimum value of an inner diameter of the hollow body [dh] is preferably smaller than the inner diameter of the tubular member [dt].

The wire is preferably not in contact with a proximal opening edge of the tubular member.

The contact member is preferably integrally formed with a part of the handle body.

The wire is preferably made of a metal and the contact member is preferably made of a resin material.

The contact member is preferably made of at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, perfluoroalkoxy fluorine resin; polyphenylene sulfide; polyetheretherketone; polyacetal; polyamide resin; polyolefin resin; polyetherpolyamide resin; polyurethane resin; polyimide resin; and polyester resin.

A surface of the contact member including the contact part with the wire is preferably curved.

The catheter preferably further comprises a wire restriction disposed on the proximal side of the contact member.

The wire restriction is preferably integrally formed with a part of the handle body.

The wire restriction is preferably a ring.

An inner diameter of the wire restriction [dr] is preferably larger than the inner diameter of the tubular member [dt].

The wire is preferably made of a metal and the wire restriction is preferably made of a resin material.

The wire restriction is preferably made of at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, perfluoroalkoxy fluorine resin; polyphenylene sulfide; polyetheretherketone; polyacetal; polyamide resin; polyolefin resin; polyetherpolyamide resin; polyurethane resin; polyimide resin; and polyester resin.

### Effects of the Invention

The catheter o the present invention can control the degree of bending of the catheter tube at the distal side and can make the wires located in the catheter handle less likely to be damaged or broken.

### BRIEF DESCRIPTION OF THE DRAWINGS

FIG. 1 is an overall view of a catheter equipped with a catheter handle according to an embodiment of the present invention.

FIG. 2 is a plan view of a major part of the catheter handle provided to the catheter shown in FIG. 1.

FIG. 3 is a plan view (part of which is a cross-section view) of an inner structure of the major part of the catheter handle shown in FIG. 2.

FIG. 4 is a plan view (part of which is a cross-section view) of an inner structure of the major part of the catheter handle shown in FIG. 2, when a rotary member is rotated.

FIG. 5A is a cross-section view of a tubular member, a contact member, and a wire, which shows an inner structure of a distal side of a catheter handle according to one embodiment of the present invention, and FIG. 5B is a V-V cross-section view of the contact member shown in FIG. 5A.

FIG. 6A is a cross-section view of a tubular member, a contact member, and a wire, which shows an inner structure of a distal side of a catheter handle according to another embodiment of the present invention, and FIG. 6B is a VI-VI cross-section view of the contact member shown in FIG. 6A.

FIG. 7A is a cross section view of a tubular member, a contact member, and a wire, which shows an inner structure of a distal side of a catheter handle according to yet another embodiment of the present invention, and FIG. 7B is a VII-VII cross-section view of the contact member shown in FIG. 7A.

FIG. 8A is a cross section view of a tubular member, a contact member, and wires, which shows an inner structure of a distal side of a catheter handle according to yet another embodiment of the present invention, and FIG. 8B is a VIII-VIII cross-section view of the contact member shown in FIG. 8A.

FIG. 9A is a cross section view of a tubular member, a contact member, and wires, which shows an inner structure of a distal side of a catheter handle according to yet another embodiment of the present invention, and FIG. 9B is a IX-IX cross-section view of the contact member shown in FIG. 9A.

FIG. 10A is a cross section view of a tubular member, a contact member, and wires, which shows an inner structure of a distal side of a catheter handle according to yet another embodiment of the present invention, and FIG. 10B is a X-X cross-section view of the contact member shown in FIG. 10A.

### MODE FOR CARRYING OUT THE INVENTION

Hereinafter, the present invention will be described with reference to embodiments. The present invention, however, is not limited by the following embodiments but can also be absolutely carried out with appropriate changes to the embodiments within a scope in compliance with the intent described above and later, and all the changes are to be encompassed within a technical scope of the present invention. Note that, in each drawing, hatching, reference signs for components, and the like may be omitted for convenience of description, and in such a case, the specification and other drawings are to be referred to. Further, the dimensions of the various components in the drawings are provided for the purpose of facilitating the understanding of the feature of the present invention, and the dimensions may differ from the actual dimensions in some cases.

The present invention relates to a catheter such as an electrode catheter a catheter tube of which is equipped with electrodes. The electrode catheter is usually composed of a catheter tube inserted into body lumens such as blood vessels, and a handle provided on the proximal side of the catheter tube. At least one wire is disposed inside the catheter tube, and the distal side of the wire is fixed to the end of the catheter tube and the proximal side of the wire is fixed to the handle. Manipulating the handle to pull the wire toward the handle, the distal side of the catheter tube can be bent. The electrode catheter also has conductive wires inside the catheter tube, and the distal side of the conductive wires is connected to the electrodes provided on the distal side of the catheter tube and the proximal side of the conductive wires is connected to a connector through the handle to be connected to a detector or an electric source via connecting cables, which enables electric signals from the electrodes to be received, or electricity to be applied to the electrodes. With the electrode catheter, electrocardiogram can be measured and electrical impulse can be applied to cardiac muscle (pacing).

In the handle for the electrode catheter, the at least one wire and the conductive wires are disposed. Pulling the wire toward the handle can make the distal end of the catheter tube bend. Since the wire receives pulling force and slides along inside the catheter when the wire is pulled toward the handle, there is a problem that the wire may be damaged or broken while the wire is used repeatedly. The catheter of the present invention can make the wire less likely to be damaged or broken to improve durability of the catheter.

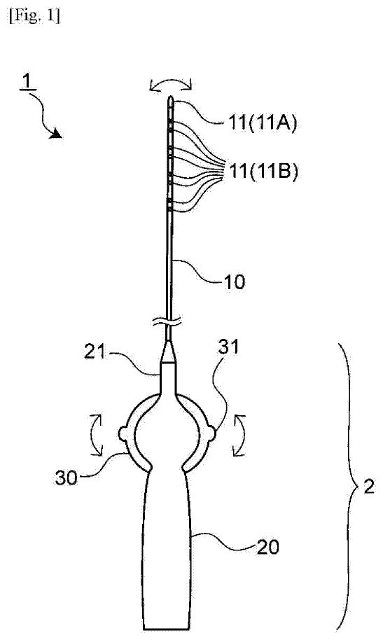

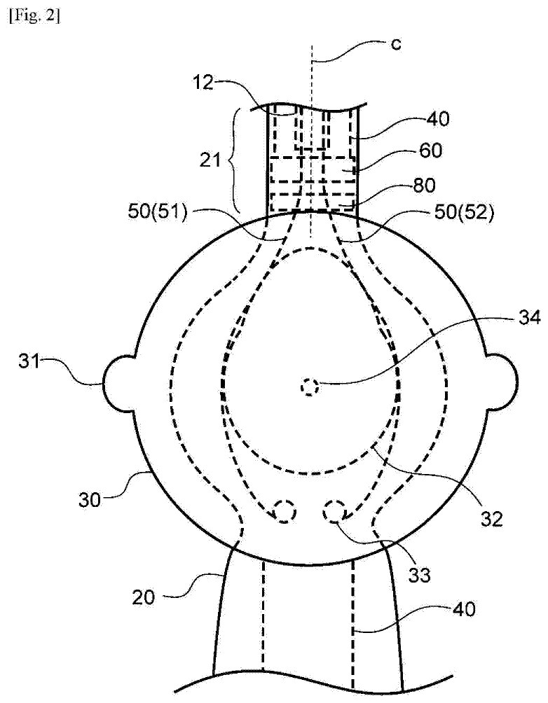

Referring to FIG. 1 and FIG. 2, a catheter according to an embodiment of the present invention will be described. FIG. 1 is an overall view of the catheter equipped with the catheter handle according to an embodiment of the present invention, and FIG. 2 is a plan view of a major part of the catheter handle provided to the catheter shown in FIG. 1. A catheter 1 has a distal side and a proximal side, and has a catheter handle 2 to which a catheter tube 10 having wire 50 therein is connected. The catheter 1 is, for example, used for examination or treatment of heart arrhythmia by delivering the catheter tube 10 to a patient's heart through blood vessels. In the present invention, the proximal side of the catheter 1 or the catheter handle 2 is the direction of the side of an user's, that is, an operator's hand in the extension direction of the catheter 1, and the distal side is the opposite direction to the proximal side, that is, the direction of an object of the treatment.

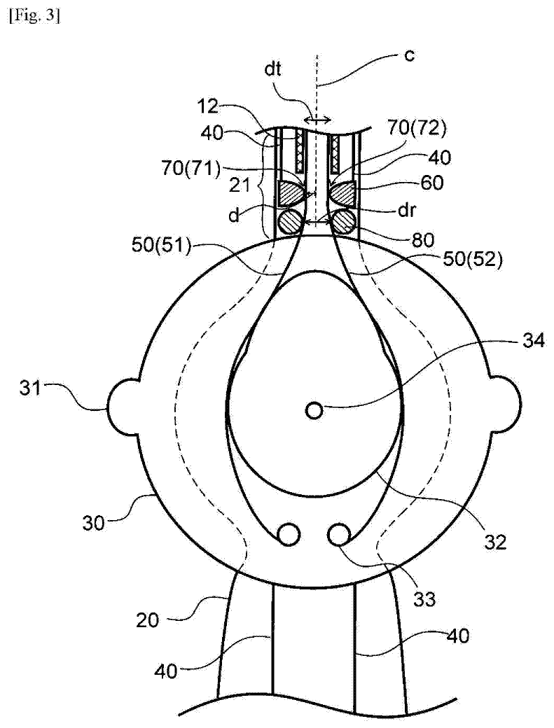

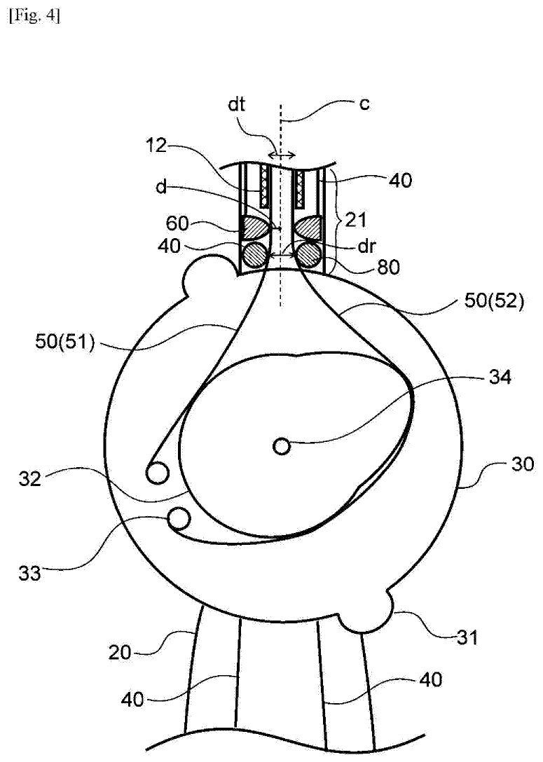

Referring to FIG. 1 to FIG. 4, the catheter handle 2 according to an embodiment of the present invention will be described. FIG. 3 is a plan view of an inner structure of a major part of the catheter handle shown in FIG. 2, part of which is cross-section views of a tubular member 12, a contact member 60, and a wire restriction 80. FIG. 4 is a plan view of the catheter handle 2 shown in FIG. 3 when a rotary member 30 is rotated, part of which is cross-section views of the tubular member 12, the contact member 60, and the wire restriction 80.

The catheter handle 2 has a handle body 20 to which the proximal side of the catheter tube 10 is connected, the rotary member 30 to which the proximal portion of the wire 50 located in the handle body 20 is fixed, a tubular member 12 having the wire inside and extending from the catheter tube 10 to the handle body 20, and a contact member 60 located more proximal than a proximal end of the tubular member and having a contact part with the wire 50.

The distal side of the wire 50 located in the catheter tube 10 is, for example, preferably fixed to a position at ⅓ from the distal end of the catheter tube 10, and the proximal portion of the wire 50 is introduced to a introducing part 21 of the handle body 20 to be fixed to the rotary member 30. The wire 50 may comprise one wire, or the wire 50 may comprise a first wire and a second wire as shown in FIG. 2 to FIG. 4.

The wire 50 is pulled to the proximal direction with a rotation of the rotary member 30, which enables the distal side of the catheter tube 10 to which the distal side of the wire 50 is fixed to bend.

The contact member 60 has the contact part with the wire 50. In a plane that is orthogonal to a central axis c of the tubular member 12 and includes a most distal contact point 70 among the contact part of the contact member 60 with the wire 50, a distance [d] between the most distal contact point 70 and the central axis c satisfies the following equation (1) in terms of an inner diameter [dt] of the tubular member 12.

[d]<[dt]/2 (1)

Since [dt] is the inner diameter of the tubular member 12, [dt]/2 is a distance between an inner wall of the tubular member 12 and the central axis c in the plane orthogonal to the central axis of the tubular member 12, which is an inner radius of the tubular member 12.

When the distance [d] satisfies the above equation (1) in terms of the inner diameter of [dt] of the tubular member 12, the wire 50 extending through the tubular member 12 to be introduced to the introducing part 21 of the handle body 20 from the proximal end of the tubular member 12 can be introduced to the rotary member 30 not spreading to more than the inner diameter of the tubular member 12 by contacting with the contact member 60. With any route by which the wire 50 is introduced, the wire 50 can pass in the range inside the inner wall of the tubular member 12 at the proximal end of the tubular member 12 to be led to the proximal side of the catheter handle 2. As the result, damage or breaking of the wire 50 is made less likely to occur, which is otherwise led by contacting with the inner wall or an opening edge of the proximal end of the tubular member 12.

Furthermore, when the wire 50 is pulled to be in the condition shown in FIG. 4 from the condition shown in FIG. 3 with a rotation of the rotary member 30, the route of the wire 50 in the catheter handle 2 is changed, however, the position of the wire 50 at the proximal end of the tubular member 12 has no effect because the wire 50 is in contact with the contact member 60. As the result, damage or breaking of the wire 50 is made less likely to occur, which is otherwise led by contacting with the inner wall or the opening edge of the proximal end of the tubular member 12.

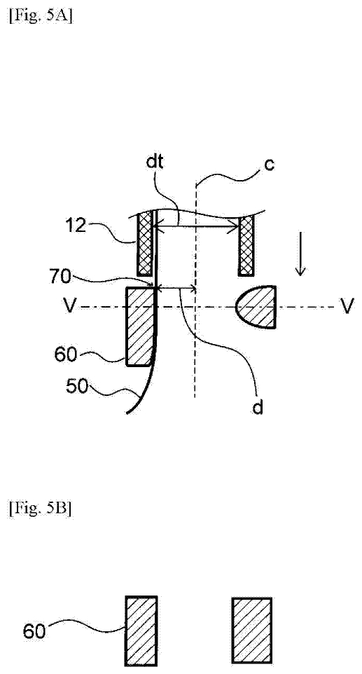

Referring to FIG. 5 to FIG. 7, the contact member 60 satisfying the above equation (1) according to embodiments of the present invention in the case where the wire 50 comprises one wire will be described.

FIG. 5 shows an example of the case where a plurality of the contact member 60 are disposed and the contact member 60 has the contact part with the wire 50 sharing a line. FIG. 5A is a cross-section view of the tubular member 12, the contact member 60, and the wire 50, and FIG. 5B is a V-V cross-section view of the contact member 60 shown in FIG. 5A. As shown in FIG. 5A, in the case where the contact member 60 has the contact part with the wire 50 sharing a line, the most distal contact point 70 is the most distal point among the contact part. In the plane that is orthogonal to the central axis c and includes the most distal contact point 70, the distance [d] between the most distal contact point 70 and the central axis c can be unambiguously determined. When the distance [d] satisfies the above equation (1), the distance [d] is smaller than the inner radius [dt/2] of the tubular member 12. As the result, the route of the wire 50 in contact with the contact member 60 is in the range inside the inner wall of the tubular member 12 at the proximal end of the tubular member 12, which makes damage or breaking of the wire 50 less likely to occur. Such damage or breaking is otherwise led by contacting with the inner wall or the opening edge of the proximal end of the tubular member 12.

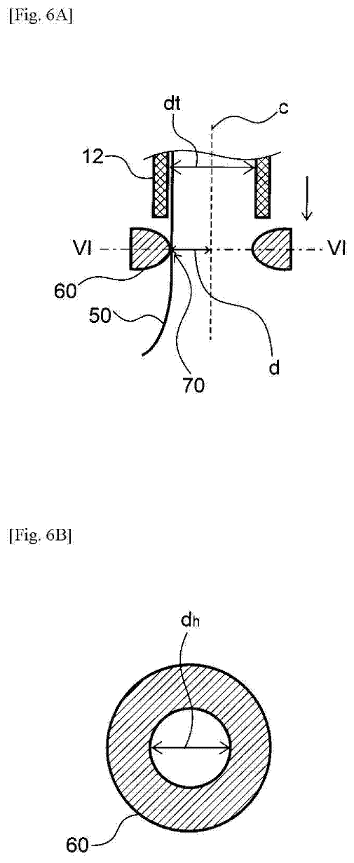

FIG. 6 shows an example of the case where the contact member 60 is a hollow body having a single hollow. FIG. 6A shows a cross-section view of the tubular member 12, the contact member 60, and the wire 50, and FIG. 6B shows a VI-VI cross-section view of the contact member 60 shown in FIG. 6A. The contact member 60 shown in FIG. 6 has the contact part with the wire 50 sharing a point. In this case, the point is the most distal contact point 70. Accordingly, in the plane that is orthogonal to the central axis c and includes the most distal contact point 70, the distance [d] between the most distal contact point 70 and the central axis c can be determined as shown in FIG. 6A, which is smaller than the inner radius [dt]/2 of the tubular member 12.

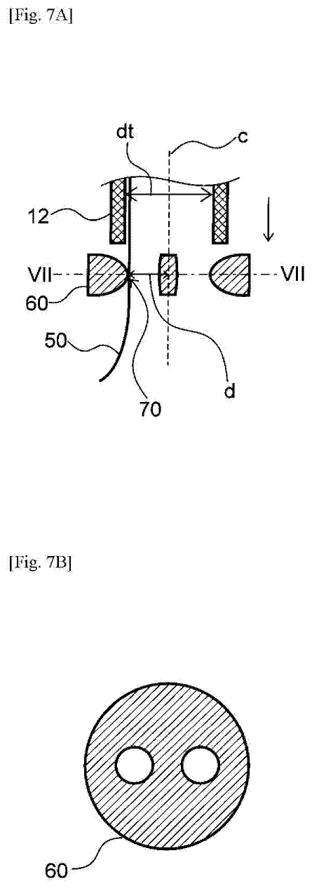

FIG. 7 shows an example of the case where the contact member 60 is a hollow body having two hollows. FIG. 7A shows a cross-section view of the tubular member 12, the contact member 60, and the wire 50, and FIG. 7B shows a VII-VII cross-section view of the contact member 60 shown in FIG. 7A. This case is similar to the case shown in FIG. 6. Regardless the number of the hollow that the contact member 60 has, in the plane that is orthogonal to the central axis c and includes the most distal contact point 70, the distance [d] between the most distal contact point 70 and the central axis c can be unambiguously determined, which is smaller than the inner radius [dt]/2 of the tubular member 12.

The contact member 60 may have any shape. In addition, other than the above example including the case where the contact member 60 has the contact part with the wire 50 sharing a line or a point, the contact member 60 may have the contact part with the wire 50 in any way, for example, sharing some points each of which is away from each other. With any shape of the contact member 60 and with any way the contact member 60 has the contact part with the wire 50, the distance [d] can be determined, and the contact member 60 of the present invention always has the distance [d] smaller than the inner radius [dt/2] of the tubular member 12. As the result, the route of the wire 50 in contact with the contact member 60 is in the range inside the inner wall of the tubular member 12 at the proximal end of the tubular member 12, which makes damage or breaking of the wire 50 less likely to occur. Such damage or breaking is otherwise led by contacting with the inner wall or the opening edge of the proximal end of the tubular member 12.

In a catheter according to an embodiment of the present invention, the wire comprises a first wire 51 and a second wire 52, and the contact member 60 has a first contact part with the first wire 51 and a second contact part with the second wire 52. In a plane that is orthogonal to the central axis c of the tubular member 12 and includes a most distal first contact point 71 among the first contact part, a distance [d] between the most distal first contact point 71 and the central axis c satisfies the above equation (1) in terms of the inner diameter [dt] of the tubular member 12, and in a plane that is orthogonal to the central axis c of the tubular member 12 and includes a most distal second contact point 72 among the second contact part, a distance [d] between the most distal second contact point 72 and the central axis c satisfies the above equation (1) in terms of the inner diameter [dt] of the tubular member 12.

[d]<[dt]/2 (1)

When the distance [d] to both the first wire 51 and the second wire 52 satisfy the above equation (1), the first wire 51 and the second wire 52 extending through the tubular member 12 to be introduced to the introducing part 21 of the handle body 20 from the proximal end of the tubular member 12 can be introduced to the rotary member 30 not spreading to more than the inner diameter of the tubular member 12 by contacting with the contact member 60. With any route by which the first wire 51 and the second wire 52 are introduced to the proximal side of the catheter handle 2, the first wire 51 and the second wire 52 can pass in the range inside the inner wall of the tubular member 12 at the proximal end of the tubular member 12 to be led to the proximal side of the catheter handle 2. As the result, damage or breaking of the first wire 51 and the second wire 52 can be made less likely to occur, which is otherwise led by contacting with the inner wall or the opening edge of the proximal end of the tubular member 12.

Furthermore, when the second wire 52 is pulled to be in the condition shown in FIG. 4 from the condition shown in FIG. 3 with a rotation of the rotary member 30, the routes of the first wire 51 and the second wire 52 in the catheter handle 2 are changed, however, the positions of the first wire 51 and the second wire 52 at the proximal end of the tubular member 12 have no effect because the first wire 51 and the second wire 52 are in contact with the contact member 60. As the result, damage or breaking of the first wire 51 and the second wire 52 can be made less likely to occur, which is otherwise led by contacting with the inner wall or the opening edge of the proximal end of the tubular member 12.

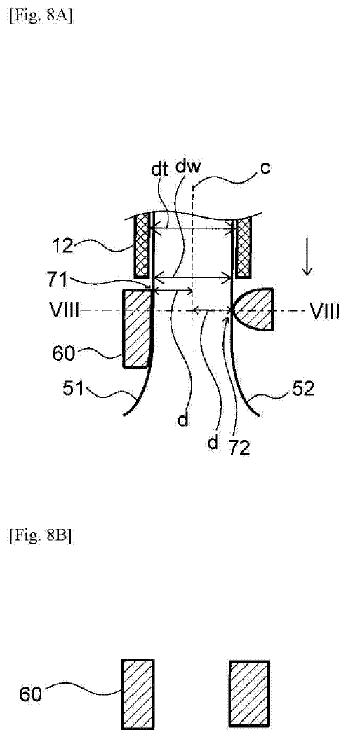

Referring to FIG. 8 to FIG. 10, the contact member 60 satisfying the above equation (1) according to embodiments of the present invention in the case where the wire 50 comprises two wires will be described.

FIG. 8 shows an example of the case where a plurality of the contact member 60 are disposed and the contact member 60 has the first contact part with the first wire 51 sharing a line and has the second contact part with the second wire 52 sharing a point. FIG. 8A is a cross-section view of the tubular member 12, the contact member 60, the first wire 51, and the second wire 52, and FIG. 8B is a VIII-VIII cross-section view of the contact member 60 shown in FIG. 8A. As shown in FIG. 8A, in the case where the contact member 60 has the first contact part with the first wire 51 sharing a line, the most distal first contact point 71 is the most distal point among the first contact part. In the plane that is orthogonal to the central axis c and includes the most distal first contact point 71, the distance [d] between the most distal first contact point 71 and the central axis c can be unambiguously determined. Since the contact member 60 has the second contact part with the second wire 52 sharing a point, which is the most distal second contact point 72, in the plane that is orthogonal to the central axis c and includes the most distal second contact point 72, the distance [d] between the most distal second contact point 72 and the central axis c can be unambiguously determined. Each of [d] is smaller than the inner radius [dt/2] of the tubular member 12. As the result, the routes of the first wire 51 and the second wire 52 in contact with the contact member 60 is in the range inside the inner wall of the tubular member 12 at the proximal end of the tubular member 12, which makes damage or breaking of the first wire 51 and the second wire 52 less likely to occur. Such damage or breaking is otherwise led by contacting with the inner wall or the opening edge of the proximal end of the tubular member 12.

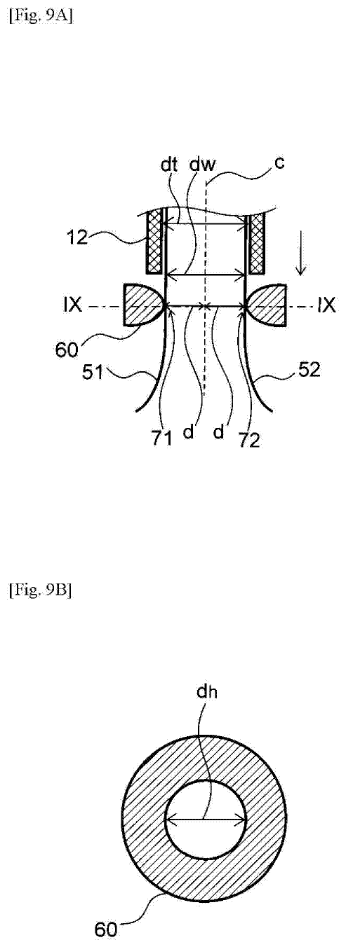

FIG. 9 shows an example of the case where the contact member 60 is a hollow body having a single hollow. FIG. 9A shows a cross-section view of the tubular member 12, the contact member 60, the first wire 51, and the second wire 52, and FIG. 9B shows a IX-IX cross-section view of the contact member 60 shown in FIG. 9A. The contact member 60 shown in FIG. 9 has the first contact part with the first wire 51 and the second contact part with the second wire 52 sharing a point respectively. In this case, each point shared by the contact member 60 and the first wire 51 and the second wire 52 is the most distal first contact point 71 and the most distal second contact point 72 respectively. Accordingly, in the plane that is orthogonal to the central axis c and includes the most distal first contact point 71, the distance [d] between the most distal first contact point 71 and the central axis c can be determined as shown in FIG. 9A, and in the plane that is orthogonal to the central axis c and includes the most distal second contact point 72, the distance [d] between the most distal second contact point 72 and the central axis c can be determined as shown in FIG. 9A, both of which is smaller than the inner radius [dt]/2 of the tubular member 12.

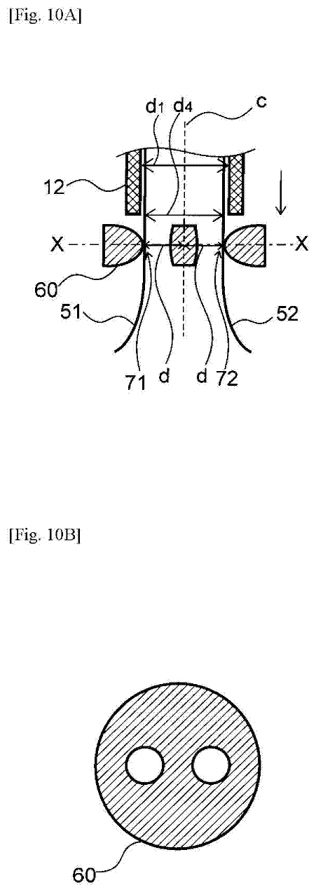

FIG. 10 shows an example of the case where the contact member 60 is a hollow body having two hollows. FIG. 10A shows a cross-section view of the tubular member 12, the contact member 60, the first wire 51, and the second wire 52, and FIG. 10B shows a X-X cross-section view of the contact member 60 shown in FIG. 10A. This case is similar to the case shown in FIG. 9. Regardless the number of the hollow that the contact member 60 has, in the plane that is orthogonal to the central axis c and includes the most distal first contact point 71, the distance [d] between the most distal first contact point 71 and the central axis c can be determined as shown in FIG. 9A, and in the plane that is orthogonal to the central axis c and includes the most distal second contact point 72, the distance [d] between the most distal second contact point 72 and the central axis c can be determined as shown in FIG. 9A, both of which is smaller than the inner radius [dt]/2 of the tubular member 12.

The contact member 60 may have any shape. In addition, the contact member 60 may have the first contact part with the first wire 51 and the second contact part with the second wire 52 in any way. With any shape of the contact member 60 and with any way the contact member 60 is in contact with the first wire 51 and the second wire 52, each of the distance [d] can be determined, and the contact member 60 of the present invention always has each of the distance [d] smaller than the inner radius [dt/2] of the tubular member 12. As the result, the routes of the first wire 51 and the second wire 52 in contact with the contact member 60 are in the range inside the inner wall of the tubular member 12 at the proximal end of the tubular member 12, which makes damage or breaking of the first wire 51 and the second wire 52 less likely to occur. Such damage or breaking is otherwise led by results from contacting with the inner wall or the opening edge of the proximal end of the tubular member 12.

The contact member 60 is preferably located more distal than a distal end of the rotary member 30, which can prevent the contact member 60 from obstructing the rotation of the rotary member 30.

The wire 50 is preferably not in contact with the proximal opening edge of the tubular member 12, so that damage or breaking of the wire 50 becomes less likely to be occur. Such damage or breaking is otherwise led by contacting with the inner wall or the opening edge of the proximal end of the tubular member 12.

In the case where the wire comprises the first wire 51 and the second wire 52, a distance [dw] between the first wire 51 and the second wire 52 at the proximal end of the tubular member 12 is smaller than the inner diameter of the tubular member [dt]. As the result, the first wire 51 and the second wire 52 are placed at a distance less than the inner diameter [dt] of the tubular member 12, and when the wires are introduced in the catheter handle 2 through the proximal end of the tubular member 12, damage or breaking of the first wire 51 and the second wire 52 can be made less likely to occur, which is otherwise led by contacting with the inner wall or the opening edge of the proximal end of the tubular member 12.

The contact member 60 is preferably a hollow body having a single hollow through which the wire 50 passes, and the minimum value of an inner diameter of the hollow body [dh] is smaller than the inner diameter of the tubular member [dt]. For example as shown in FIG. 6 and FIG. 9, the contact member 60 may be a hollow body having a single hollow, and the hollow body may have different inner diameters in the proximal/distal direction. With the minimum value of such a different inner diameter [dh] smaller than the inner diameter of the tubular member [dt], the route of the wire 50 in contact with the contact member 60 is in the range inside the inner wall of the tubular member 12 at the proximal end of the tubular member 12, which is preferable. Moreover, with any number of the wire 50, or with any disposition of the wire 50, the contact member 60 being the hollow body can easily contact with the wire 50, which is preferable.

With any shape of the contact member 60, a surface of the contact member 60 including the one or more contact part with the wire 50 is preferably curved. The curved surface of the contact member 60 including the contact part with the wire 50 can prevent stress on the wire 50 from concentrating when the wire 50 slides in contact with the contact member 60, which can make the wire 50 less likely to be worn or damaged.

The contact member 60 may be integrally formed with a part of the handle body 20. Integrally forming can reduce the number of components and processes for manufacturing the catheter handle 2, which is preferable.

The wire 50 is preferably made of a metal and the contact member 60 is preferably made of a resin material, which can makes the wire 50 less likely to be worn or damaged while being in contact with or sliding along the contact member 60.

The contact member 60 is preferably made of at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, and perfluoroalkoxy fluorine resin; polyphenylene sulfide; polyetheretherketone; polyacetal; polyamide resin; polyolefin resin; polyetherpolyamide resin; polyurethane resin; polyimide resin; and polyester resin. Among them, at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, and perfluoroalkoxy fluorine resin; polyphenylene sulfide; and polyetheretherketone is more preferable, and at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, and perfluoroalkoxy fluorine resin is still more preferable. The contact member 60 made of such a material having self-lubricating properties can reduce friction force on the wire 50 while contacting with the contact member 60, which can makes the wire 50 less likely to be worn or damaged while being in contact with or sliding along the contact member 60.

The catheter tube 10 is flexible and has an annular structure, and for example, made of synthetic resin such as polyolefin resin (such as polyethylene and polypropylene), polyamide resin (such as nylon), polyester resin (such as PET), aromatic polyether ketone resin (such as PEEK), polyetherpolyamide resin, polyurethane resin, polyimide resin, fluorine resin (such as PTFE, PFA, ETFE); and metal such as stainless steel, carbon steel, nickel-titanium alloy. The metals may be used for metal wire embedded in the tube made of synthetic resin. The length in the axial direction (the proximal/distal direction) of the catheter tube 10, which is a few times to several tens of times as long as the catheter handle 2, is, for example, about 500 mm to 1200 mm. The outer diameter of the catheter tube 10 may be, for example, about 1.0 mm to 3 mm.

A plurality of electrodes 11 may be disposed on the distal side of the catheter tube 10. In FIG. 1, a tip electrode 11A and a plurality of ring electrodes 11B are disposed. With the electrode catheter, the electrodes 11 are connected with the inside of a patient's heart to inspect cardiac dysrhythmia. The electrodes 11 may be made of metal materials such as copper, gold, platinum, aluminum, iron, or an alloy thereof. In order to make the catheter sensitive to radiographic visualization while being used, the electrodes 11 are preferably made of platinum or an alloy thereof.

The wire 50 may be made of metal such as stainless steel, carbon steel, nickel-titanium alloy, or synthetic resin such as polyamide resin (such as nylon), polyolefin resin (such as polyethylene and polypropylene), polyester resin (such as PET), aromatic polyether ketone resin (such as PEEK), polyetherpolyamide resin, polyurethane resin, polyimide resin, fluorine resin (such as PTFE, PFA, ETFE). The wire 50 may comprises a plurality of wires, each of which may be a wire or has a structure including a plurality of striate bodies. The diameter of the wire 50 may be preferably 50 μm or more, more preferably 80 μm or more, and still more preferably 100 μm or more. The diameter of the wire 50 may be preferably 300 μm or less, more preferably 400 μm or less, and still more preferably 500 μm or less.

The tubular member 12 in which the wire 50 is disposed extends from the catheter tube 10 to the distal side of the introducing part 21 of the handle body 20 to introduce the wire 50 into the handle body 20. Since the wire 50 is covered by the tubular member 12 in the catheter tube 10, the plurality of wires, or the wire and the conductive wires are less likely to be entangled with each other, and as a result, the wire is less likely to be damaged or broken. The tubular member 12 may be made of metal materials, and may have a mesh structure or a coil structure to have flexibility.

The inner diameter of the tubular member [dt] is not limited as long as it is possible for the wire 50 to extend through the tubular member, and preferably 0.2 mm or more, more preferably 0.4 mm, and still more preferably 0.6 mm. The inner diameter of the tubular member [dt] is preferably 2.0 mm or less, more preferably 1.5 mm or less, and still more preferably 1.0 mm or less.

The wire restriction 80 may be disposed on the proximal side of the contact member 60. The wire restriction 80 is preferably a ring. The wire restriction 80 restricts the route of the wire 50 when the wire 50 is in contact with the contact member 60 and then introduced into the proximal side of the catheter handle 2, which prevent the wire 50 from unnecessary spreading to make contact with an unintended portion of the catheter handle 2. The wire restriction 80 is preferably made of resin materials.

The wire restriction 80 may be integrally formed with a part of the handle body 20. Integrally forming can reduce the number of components and processes for manufacturing the catheter handle 2, which is preferable.

The wire restriction 80 may be a ring. The ring structure is preferable because the wire restriction 80 can restrict the route of the wire 50 in all directions regardless of the number of the wire 50 and how the wire 50 is placed.

In the case where the wire restriction 80 is a ring, the inner diameter of the wire restriction 80 [dr] is preferably larger than the inner diameter of the tubular member 12 [dt], more preferably 1.5 times or more the inner diameter [dt], and still more preferably 2 times or more the inner diameter [dt]. Such a lower limit of the inner diameter [dr] make it easy for the wire 50 to be introduced to the rotary member 30 and a wire guide member 32. The inner diameter of the wire restriction 80 [dr] is preferably 10 times or less the inner diameter [dt], more preferably 9 times or less the inner diameter [dt], and still more preferably 9 times or less the inner diameter [dt]. Due to such a higher limit of the inner diameter [dr] the route of the wire 50 can be effectively restricted not to extend unnecessarily.

The wire 50 is preferably made of a metal and the wire restriction 80 is preferably made of a resin material, which can makes the wire 50 less likely to be worn or damaged while being in contact with or sliding along the wire restriction 80.

The wire restriction 80 is preferably made of at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, and perfluoroalkoxy fluorine resin; polyphenylene sulfide; polyetheretherketone; polyacetal; polyamide resin; polyolefin resin; polyetherpolyamide resin; polyurethane resin; polyimide resin; and polyester resin. Among them, at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, and perfluoroalkoxy fluorine resin; polyphenylene sulfide; and polyetheretherketone is more preferable, and at least one selected from the group consisting of fluorine resin such as polytetrafluoroethylene, tetrafluoroethylene-hexafluoropropylene copolymer, ethylene-tetrafluoroethylene copolymer, ethylene-chlorotrifluoroethylene copolymer, and perfluoroalkoxy fluorine resin is still more preferable. The wire restriction 80 made of such a material having self-lubricating properties can reduce friction force on the wire 50 while being in contact with the contact member 60, which can makes the wire 50 less likely to be worn or damaged while being contact with or sliding along the wire restriction 80.

The rotary member 30 may have a rotation axis 30 orthogonal to both the proximal/distal direction and the width direction of the catheter handle 2, and is preferably configured such that the rotary member 30 can revolve around the rotation axis 30 on the plane formed by both the proximal/distal direction and the width direction. Also, the rotary member 30 may, as necessary, shift in the catheter handle 2.

The rotary member 30 is preferably equipped with a wire guide member 32. The wire guide member 32 may be attached to the rotary member 30 directly or indirectly. The wire guide member 32 may be integrally formed with the rotary member 30 or may be partially fixed to the rotary member 30 so that the wire guide member 32 and the rotary member 30 rotate together, or the wire guide member 32 may be formed not to rotate with the rotary member 32. The catheter handle 2 shown in FIG. 2 to FIG. 4 is equipped with the wire guide member 32 the rotation axis of which is the same as the rotary member 32.

The wire restriction 80 may be disposed on the distal side of the distal end of the wire guide member 32.

The wire 50 may be disposed so as to be in contact with the outer edge of the wire guide member 32 so that the wire guide member 32 regulates the route of the wire 50 in the rotary member 30. When the rotary member 30 is rotated, the wire guide member 32 makes the route of the wire 50 change to pull the wire 50 located in the tube 10. For example, in the case where the wire guide member 32 has longer length in the proximal/distal direction than the width direction as shown in FIG. 3 and FIG. 4, the wire 52 shown in FIG. 4 where the wire guide member 32 is rotated is being pulled in the width direction of the catheter handle 2, comparing to the wire 52 shown in FIG. 3 where the wire guide member 32 is not rotated. In such a condition, since the wire 52 is in contact with the contact member 60 satisfying the equation (1), the route of the wire 52 is in the range inside the inner wall of the tubular member 12 at the proximal end of the tubular member 12. As the result, damage or breaking of the wire 52 is made less likely to occur, which is otherwise led by contacting with the inner wall or an opening edge of the proximal end of the tubular member 12.

An operating part 31 may be provided for easy operation of the rotary member 30. The operating part 31 may be integrally formed with the rotary member 30 to form a protrusion or a recess from the rotary member 30. The operating part 31 may comprise a plurality of operating parts, and may be provided to the wire guide member 32.

The rotary member 30 is preferably equipped with a wire locking member 33 such that the proximal side of the wire 50 is fixed to the wire locking member 33. The wire is fixed to the rotary member 30 by, for example, gluing with adhesive, welding with synthetic resin, fixing with another parts such as screws, concavo-convex fitting, and a combination thereof. The rotary member 30 may be made of, for example, synthetic resin. The rotary member 30 is preferably formed as a housing in which the proximal side of the wire 50 is placed.

The catheter 1 according to an embodiment of the present invention also has a conductive wire 40 disposed in the catheter tube 10. A distal portion of the conductive wire 40 is fixed to the catheter tube 10 and a proximal portion of the conductive wire 40 extends to the proximal side of the rotary member 30. The catheter handle 2 can make the wire 50 and the conductive wire 40 less likely to interfere with each other, because the wire 50 extends through the tubular member 12 disposed in the catheter tube 10, which can make the wire 50 and the conductive wire 40 less likely to be damaged or broken in the catheter tube 10.

The distal side of the conductive wire 40 is connected to an electrode provided on the distal side of the catheter tube, and the proximal side of the conductive wire 40 is connected to a connector through the catheter handle 2 to be connected to a detector or an electric source via connecting cables. The conductive wire 40 may comprises a conductive wire or a plurality of conductive wires. In general, the conductive wire 40 comprises a plurality of conductive wires. The plurality of conductive wires 40 may be separately placed in different lumens, or the plurality of conductive wires 40 may be placed in the same lumen. Or the plurality of conductive wires 40 can be bundled to be placed.

The conductive wire 40 just has to include at least a conductive material including iron wire, silver wire, stainless wire, copper wire, tungsten wire, nickel titanium wire, and an alloy thereof. The conductive wire 40 preferably includes the conductive material as a core material covered by an insulating material. The insulating material may include fluorine resin (such as PTFE, PFA, FEP, and ETFE), polyolefin resin (such as polyethylene and polypropylene), and polyvinyl chloride resin.

### DESCRIPTION OF REFERENCE SIGNS

1: catheter
2: handle
10: catheter tube
11, 11A, 11B: electrode
12: tubular member
20: handle body
21: introducing part
30: rotary member
31: operating part
32: wire guard member
33: wire locking part
34: rotation axis of the rotary member
40: conductive wire
50: wire
51: first wire
52: second wire
60: contact member
70: most distal contact point
71: most distal first contact point
72: most distal second contact point
80: wire restriction

1. A catheter, comprising:
a catheter tube having a distal side and a proximal side and provided with at least one wire therein; and
a catheter handle connected to the proximal side of the catheter tube, the catheter handle comprising a handle body connected to the proximal side of the catheter tube, a rotary member fixed to a proximal portion of the wire and disposed in the handle body, a tubular member having the wire inside and extending from the catheter tube to the handle body, and a contact member having a contact part with the wire and located more proximal than a proximal end of the tubular member, wherein
in a plane that is orthogonal to a central axis of the tubular member and includes a most distal contact point among the contact part, a distance (d) between the most distal contact point and the central axis satisfies the following equation (1) in terms of an inner diameter of the tubular member (dt):
(d)<dt/2 (1).

2. The catheter according to claim 1, wherein
the contact member is located between the proximal end of the tubular member and a distal end of the rotary member.

3. The catheter according to claim 1, wherein
said at least one wire comprises a first wire and a second wire,
the contact member has a first contact part with the first wire and a second contact part with the second wire,
in a plane that is orthogonal to the central axis of the tubular member and includes a most distal first contact point among the first contact part, a distance (d) between the most distal first contact point and the central axis satisfies the above equation (1) in terms of the inner diameter of the tubular member (dt), and
in a plane that is orthogonal to the central axis of the tubular member and includes a most distal second contact point among the second contact part, a distance (d) between the most distal second contact point and the central axis satisfies the above equation (1) in terms of the inner diameter of the tubular member (dt).

4. The catheter according to claim 3, wherein
a distance (dw) between the first wire and the second wire at a proximal end of the tubular member is smaller than the inner diameter of the tubular member (dt).

5. The catheter according to claim 1, wherein the contact member is a hollow body having a single hollow, and a minimum value of an inner diameter of the hollow body (dh) is smaller than the inner diameter of the tubular member (dt).

6. The catheter according to claim 1, wherein the wire is not in contact with a proximal opening edge of the tubular member.

7. The catheter according to claim 1, wherein the contact member is integrally formed with a part of the handle body.

8. The catheter according to claim 1, wherein the wire is made of a metal and the contact member comprises a resin material.

9. The catheter according to claim 1, wherein the contact member comprises at least one material selected from the group consisting of fluorine-based resin, polyphenylene sulfide; polyetheretherketone; polyacetal; polyamide resin; polyolefin resin; polyetherpolyamide resin; polyurethane resin; polyimide resin; and polyester resin.

10. The catheter according to claim 1, wherein a surface of the contact member including the contact part with the wire is curved.

11. The catheter according to claim 1, further comprising a wire restriction disposed between the contact member and a distal end of the rotary member.

12. The catheter according to claim 11, wherein the wire restriction is integrally formed with the handle body as a part of the handle body.

13. The catheter according to claim 11, wherein the wire restriction is a ring shaped member.

14. The catheter according to claim 13, wherein an inner diameter of the wire restriction (dr) is larger than the inner diameter of the tubular member (dt).

15. The catheter according to claim 11, wherein the wire comprises a metal and the wire restriction comprises a resin material.

16. The catheter according to claim 15, wherein the wire restriction comprises at least one resin material selected from the group consisting of fluorine-based resin, polyphenylene sulfide; polyetheretherketone; polyacetal; polyamide resin; polyolefin resin; polyetherpolyamide resin; polyurethane resin; polyimide resin; and polyester resin.

17. The catheter according to claim 1, further comprising an electrode disposed at a distal portion of the catheter tube, and a conductive wire extending from the catheter tube to the proximal side of the rotary member, wherein
a distal side of the conductive wire is electrically connected to the electrode.
